(19) United States
(12) Patent Application Publication
(10) Pub. No.: US 2005/0042267 A1
(43) Pub. Date: Feb. 24, 2005

(54) System for providing therapy to a portion of a body

(75) Inventor: Ricky W. Purcell, Alpharetta, GA (US)

Correspondence Address: Schwegman, Lundberg, Woessner & Kluth, P.A., P.O. Box 2938, Minneapolis, MN, 55402, US

(73) Assignee: Kimberly-Clark Worldwide, Inc.

(21) Appl. No.: 10/645,447

(22) Filed: Aug. 20, 2003

(51) Int. Cl. A61L 15/16; A61F 13/02

(52) U.S. Cl. 424/448

(57) ABSTRACT

A system and method for providing therapy to a portion of a body that includes a holder and a first patch which is held by the holder. The first patch applies a first therapy to the portion of the body when the first patch is placed near the portion of the body. The system further includes a second patch that is held by the holder. The second patch applies a second therapy to the portion of the body when the second patch is placed near the portion of the body. The method includes selecting a patch from a holder that holds a plurality of patches and applying the patch near the portion of the body. At least one of the patches held by the holder is adapted to apply a different type of therapy than the other patches such that a user or therapist is able to select from more than one therapy.

### FIELD

This invention relates in general to a system for providing therapy to a body, and in particular to a system that includes patches which provide different kinds of therapy to an injured portion of a body.

### BACKGROUND

Applying therapy to various parts of a body is a recognized practice for curing or alleviating multiple kinds of physical problems. One example therapy involves cooling an injured portion of a body by applying ice packs near the injured portion of the body in an effort to reduce swelling, inflammation and/or muscle pain.

Another cooling method utilizes a cold pack that generates cooling via an endothermic chemical reaction which takes place within the cold pack. The cold pack typically includes an solute and a liquid that are both stored within a common enclosure. The liquid and solute are initially segregated from another within the enclosure and then mixed within the enclosure to form an endothermic solution that reduces the temperature of the cold pack.

Another example therapy utilizes heat to treat symptoms such as stiffness, muscle pain, cold hands and feet, lumbago, rheumatism and neuralgia (among others). Some known heat-treating methods include direct application of heat to the body using items such as a towel, jelly and/or paste. Another example heating therapy includes applying a heating pad to an injured portion of a body to alleviate discomfort caused by injury (e.g., muscular strain).

Some heat-treating methods utilize heat patches to apply heat to a body. One such heat patch generates heat via an exothermic chemical reaction that takes place within the heat patch. Heat patches that generate heat using an exothermic reaction typically include an enclosure and a heating composition stored within the enclosure. At least a portion of the enclosure is air-permeable such that exposing the heating composition within the heat patch to air generates a heat-producing exothermic reaction.

Another therapy utilizes infrared energy reflectors to reflect infrared energy emitted by the body back into an injured portion of a body. The infrared energy reflectors are sometimes part of a patch that includes a reflective layer.

Some other therapies include applying an analgesic (for pain) or an antibiotic (for infection) to the injured portion of the body. The analgesic and/or antibiotic may be incorporated into a pack/patch that is applied directly or indirectly to the body.

Electrical stimulation may also be used to apply therapy to an injured portion of a body. The electrical stimulation is typically applied by a device that is positioned on or near the injured portion of the body. Some of the electrical devices that are used to apply electrical stimulation include their own power supply while others must be connected to an external power supply.

Another method of applying therapy to an injured area of a body includes using a flexible wrap to support the injured area of the body. The flexible wrap may also be used to hold packs/patches against the injured area of the body as the flexible wrap is wrapped around the body.

There is a need for systems and methods that are able to conveniently apply different types of therapy to an injured portion of a body. The system and method should also be capable of allowing a user or therapist to adjust or change the therapy being applied to the injured portion of the body.

### SUMMARY OF THE INVENTION

The present invention relates to a system and method for providing therapy to a portion of a body. The system and method allow a user or therapist to conveniently apply one or more alternative therapies to an injured portion of a body. The system and method also reduce the need to purchase and store excessive inventories of therapy-related products.

In one aspect, the system includes a holder and a first patch that is held by the holder. The first patch applies a first therapy to the portion of the body when the first patch is placed near the portion of the body. The system further includes a second patch that is held by the holder. The second patch applies a second therapy to the portion of the body when the second patch is placed near the portion of the body. The system makes it easier for a user or therapist to readily choose an appropriate therapy for an injured portion of a body.

In another aspect, the present invention relates to a kit of parts for providing therapy to a portion of a body. The kit includes a first patch that is adapted to apply a first therapy to the portion of the body when the first patch is placed near the portion of the body, and a second patch that is adapted to apply a second therapy to the portion of the body when the second patch is placed near the portion of the body. The kit further includes a holder that is adapted to hold the first and second patches. The kit provides a user or therapist with the ability to provide more than one type of therapy to the injured area of the body. Therefore, the kit reduces the need to stock (i.e., inventory) the multiple systems, containers, boxes, etc. that would normally be necessary to provide the ability to apply more than therapy.

In yet another aspect, the present invention relates to a method of providing therapy to a portion of a body. The method includes selecting a patch from a holder that holds a plurality of patches and applying the patch near the portion of the body. At least one of the patches that is held by the holder is adapted to apply a different type of therapy to the portion of the body than the other patches such that a user or therapist is able to select from more than one therapy. In some sample forms of the method, applying the patch near the portion of the body includes applying the patch to the portion of the body.

The purposes and features of the present invention will be set forth in the description that follows. Additional features of the invention will be realized and attained by the product and processes particularly pointed out in the written description and claims hereof, as well as from the appended drawings.

It is to be understood that both the foregoing general description and the following detailed description are exemplary and are intended to provide further explanation of the invention claimed. The accompanying drawings, which are incorporated in and constitute part of this specification, are included to illustrate and provide a further understanding of the invention.

### BRIEF DESCRIPTION OF THE DRAWINGS

The present invention will be more fully understood, and further features will become apparent, when reference is made to the following detailed description and the accompanying drawings. The drawings are merely representative and are not intended to limit the scope of the claims. Like parts depicted in the drawings are referred to by the same reference numerals.

FIG. 1 illustrates a perspective view of a system for providing therapy to a portion of a body.

FIG. 2 illustrates a perspective view of a patch from the system of FIG. 1 mounted near an injured portion of a body.

FIG. 3 illustrates a perspective view of a plurality of the patches joined together.

FIG. 4 illustrates a perspective view of a patch that may be used in the system of FIG. 1.

FIG. 5 illustrates a perspective view of another system for providing therapy to a portion of a body.

FIG. 6 illustrates a perspective view of a kit for providing therapy to a portion of a body.

### DETAILED DESCRIPTION OF THE INVENTION

In the following detailed description, reference is made to the accompanying drawings, which show specific embodiments in which the invention may be practiced. These embodiments are described in sufficient detail to enable those skilled in the art to practice the invention. It is to be understood that other embodiments may be utilized and structural changes made, such that the following detailed description is not to be taken in a limiting sense.

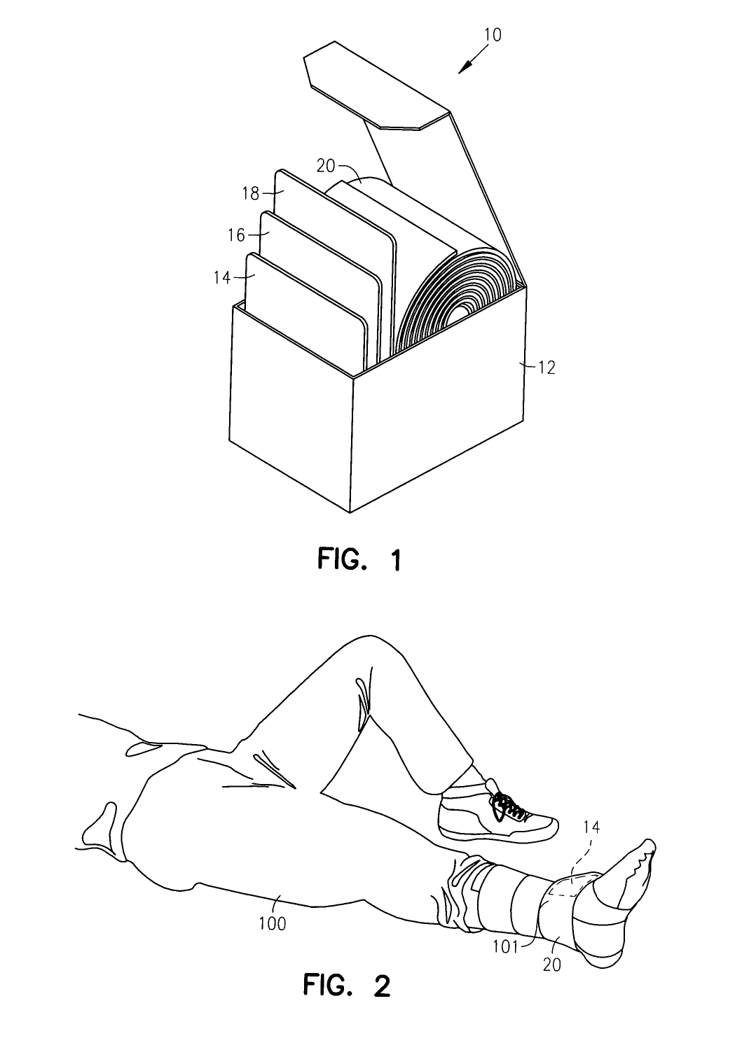

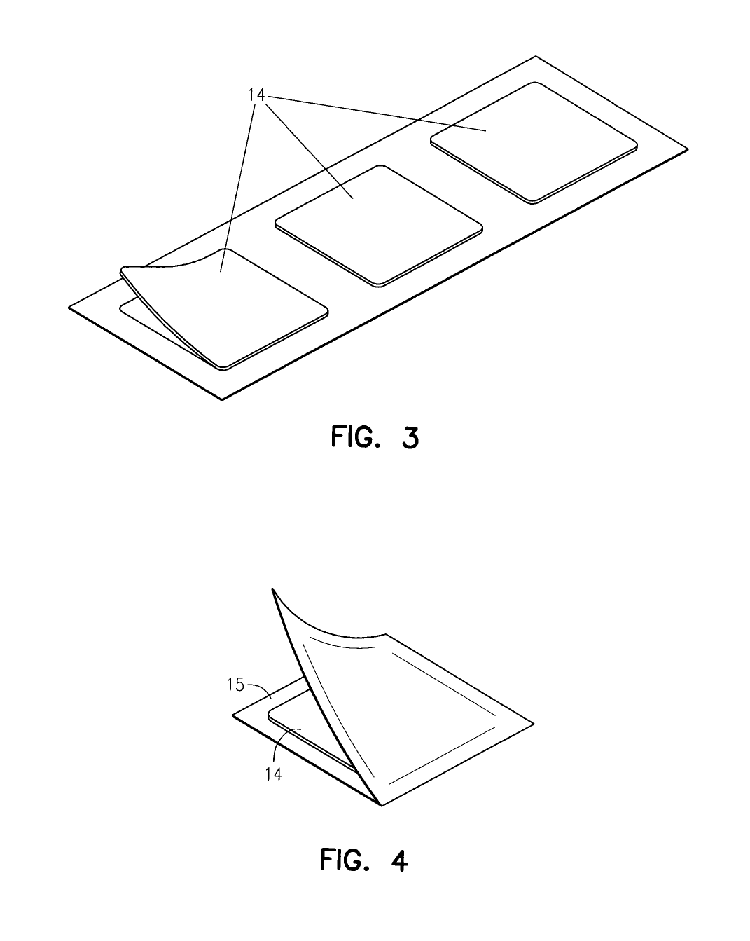

FIG. 1 illustrates a system 10 for providing therapy to a portion of a body. The system 10 includes a holder, such as container 12, and a first patch 14 that is held by the container 12. As shown in FIG. 2, the first patch 14 applies a first therapy to the portion 101 of the body 100 when the first patch 14 is placed on, or near, the portion 101 of the body 100. The system 10 further includes a second patch 16 that is held by the container 12. The second patch 16 applies a second therapy to the portion 101 of the body 100 when the second patch 16 is placed near the portion 101 of the body 100. It should be noted that second patch 16 may be applied near the portion 10 1 of the body 100 instead of the first patch 14. The choice of which patch (14 or 16) to apply will depend on the type of therapy required by the injured portion 101.

Although FIG. 1 illustrates a box being used as container 12, it should be noted that any type of container may used in the system 10. Some other example containers include cans, jars and pouches (among others).

In the sample system 10 illustrated in FIG. 1, a third patch 18 is held by the container 12. The third patch 18 applies a third therapy to the portion 101 of the body 100 when the third patch 18 is placed near the portion 101 of the body 100. Although three patches 14, 16, 18 are shown in FIG. 1, the number and type of patches may vary depending on the type of system. As an example, the first patch 14 may be an exothermic heating patch, the second patch 16 may an endothermic cooling patch, and the third patch 18 may be a topical or transdermal analgesic patch.

It should be noted that any type of patch may used in system 10 or any of the systems described herein. Some other example patches include infrared energy reflectors, antibiotic patches, electrical stimulating devices and devices that deliver accupressure therapy. As used herein, patch is meant to refer to packs, packages, inserts, buttons and/or any other device that is readily applied near an injured portion of a body to apply therapy.

The system 10 may also include a plurality of any one type of patch as long as there are at least some patches that apply different types of therapy. As shown in FIG. 3, a plurality of first patches 14 may be joined together such that the plurality of first patches 14 could be stored in container 12 with one or more the other types of patches.

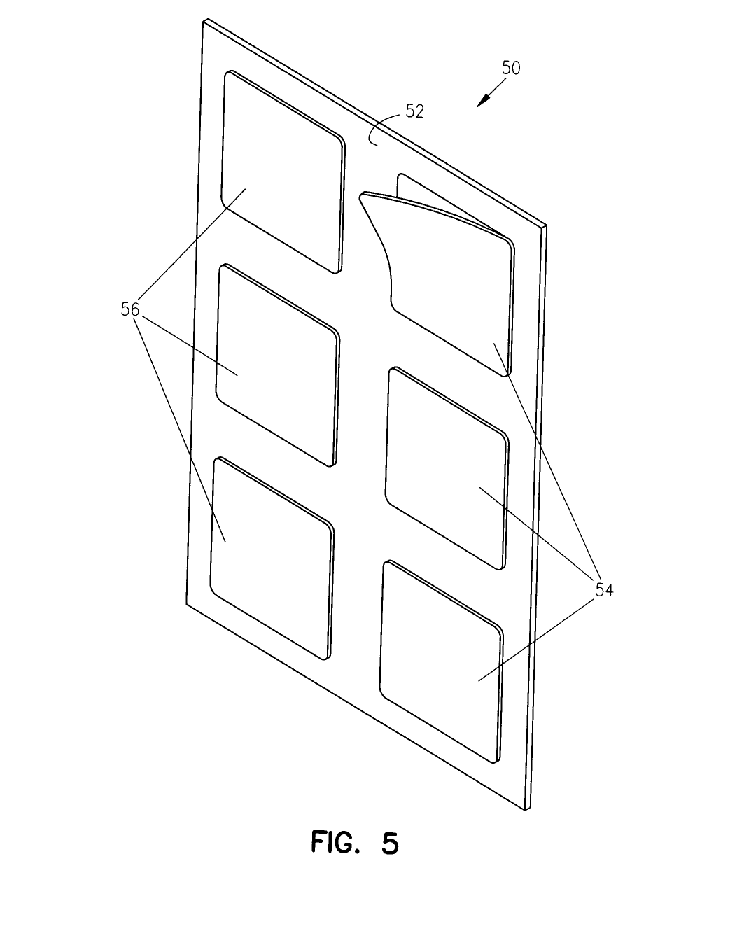

FIG. 4 shows an example patch 14 sealed inside an air-tight enclosure 15. It should be noted that some, none or all of the patches may be sealed inside some type of enclosure before they are held by the container 12. The need to seal a patch inside an enclosure will depend on the type of patch. As an example, patch 14 may be a heat patch that generates heat via an exothermic reaction. Heat patches typically include a heating composition that must be exposed to air to begin the exothermic reaction. The heating composition within such heat patches is usually exposed to air by removing the heat patch from an air-tight enclosure.

Referring again to FIGS. 1 and 2, the system 10 may further include a flexible wrap 20 for supporting a portion 101 of a body 100 and securing one or more of the patches 14, 16, 18 near the portion 101 of the body 100. The flexible wrap 20 is secured relative to the body 100 at a location near the injured portion 101 of the body. The pack 14 (and/or other packs) is then placed against the injured portion 101 of the body 100. The pack 14 is held in place by wrapping the flexible wrap 20 around the injured portion 101 of the body 100. The flexible wrap 20 is spiraled around the body 100 until an end of the flexible wrap 20 is secured to an already-wrapped section of the flexible wrap 20.

The flexible wrap 20 may be formed from one or more of layers. The number and type of layers will depend on the application where the flexible wrap 20 is used. As an example, some layers may be more elastic while other layers may be made of softer and/or more insulating materials. The proper size and shape of the flexible wrap 20 will depend on the application where the flexible wrap 20 is used, especially the size and shape of the injured portion 101 on the body 100. It should be noted that more than one size and type of flexible wrap 20 maybe included in system 10.

FIG. 5 illustrates another system 50 for providing therapy to a portion of a body. The system 50 includes a holder, such as board 52, and a plurality of first patches 54 that are releasably secured to the board 52. The first patches 54 apply a first type of therapy to the portion of the body when they are placed on, or near, the portion of the body. The system 50 further includes a plurality of second patches 56 that are also releasably secured to the board 52. The second patches 56 apply a second therapy to the portion of the body when they are placed near the portion of the body.

In some forms, system 50 may include a third patch that is releasably secured to the board 52. The third patch would apply yet another therapy to the portion 101 of the body 100 when the third patch is placed near the portion 101 of the body 100. It should be noted that the system 50 may include one or more of any one type of patch as long as there are at least some patches that apply different types of therapy.

The patches 54, 56 may be applied to the board using an adhesive such that the patches 54, 56 can be peeled from the board 52 and applied near the injured portion of the body. The decision as to the number and type of patches to apply will depend on the type of therapy required by the injured portion of the body.

A method of supporting a portion 101 of a body 100 is described herein with reference to FIGS. 1-5. The method includes selecting a patch (e.g., patch 14) from a holder (e.g., container 12) that holds a plurality of patches 14, 16, 18 with at least one of the patches adapted to apply a different type of therapy to the portion of the body than the other patches. The method further includes applying the patch 14 on, or near, the portion 101 of the body 100 (FIG. 2). It should be noted that method may further include determining an appropriate therapy to provide to the portion 101 of the body 100.

In some sample forms of the method, selecting a patch from a holder that holds a plurality of patches includes (i) selecting a heat patch (e.g., patch 14) from the plurality of patches; (ii) removing the patch (e.g., patch 14) from a container 12; and/or (iii) releasing the patch (e.g., patch 54) from a board 52 (FIG. 5). In addition, applying the patch 14 near the portion 101 of the body 100 may include wrapping the patch 14 within a flexible wrap 20 that is wrapped or spiraled around the injured portion 101 of the body 100 (FIG. 2).

The method may further include selecting at least two patches from the holder where one of the patches provides a different type of therapy than the other patches, and applying the patches near the portion of the body to provide a combined therapy. One example includes applying one or more topical analgesic patches in combination with one or more transdermal analgesic patches. Another example includes applying one or more heat patches in combination with one or more transdermal analgesic patches.

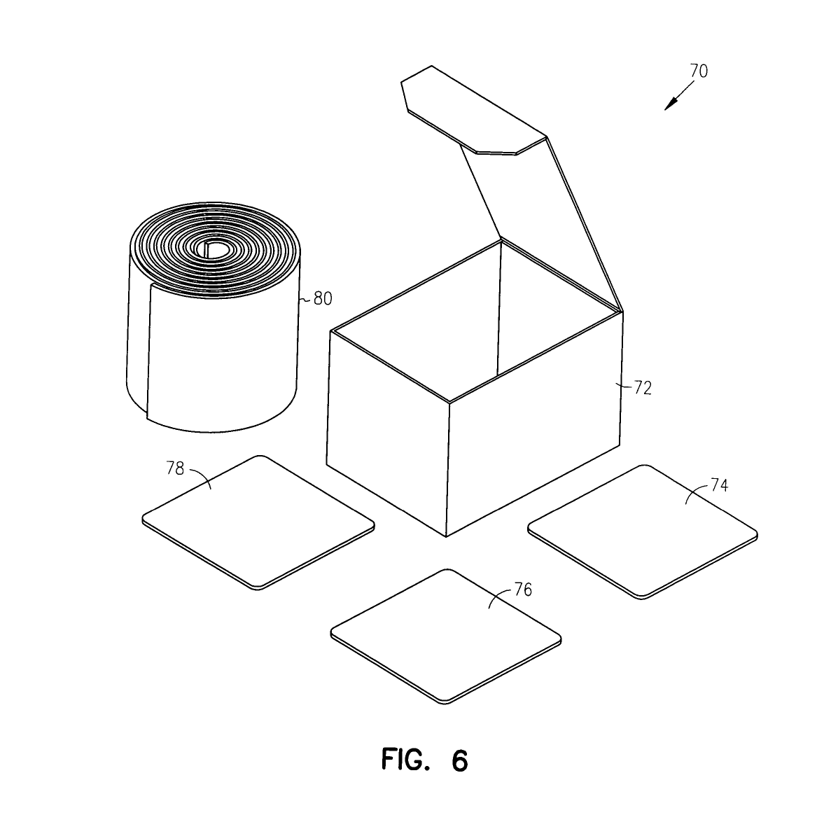

FIG. 6 shows a kit 70 of parts for providing therapy to a portion of a body. The kit includes a first patch 74 that is adapted to apply a first therapy to the portion of the body when the first patch 74 is placed near the portion of the body. The kit 70 also includes a second patch 76 that is adapted to apply a second therapy to the portion of the body when the second patch 76 is placed near the portion of the body. The kit 70 further includes a holder, such as container 72, which is adapted to hold the first and second patches 74, 76.

The kit 70 may further include a third patch 78 (e.g., an electrical stimulating device) that is adapted to apply a third therapy to the portion of the body when the third patch 78 is placed near the portion of the body. In some forms of the kit 70, the holder is a board (see, e.g., board 52 in FIG. 5) and the patches 74, 76, 78 are releasably attached to the board.

The patches used in the kit may be of any number and type of patch as long as there are at least some patches that apply different types of therapy compared to the other patches. As an example, first patch 74 may be an exothermic patch adapted to heat to the body and the second patch 76 may be an endothermic patch adapted to cool the body. In addition, one or more of the patches that are used in the kit 70 may be sealed inside an enclosure (e.g., an air-tight enclosure).

The kit 70 may further include a flexible wrap 80 that is adapted to secure one or more the patches 74, 76, 78 in the kit near the portion of the body. The proper size and shape of the flexible wrap 80 will depend on the application where the flexible wrap 80 is used (i.e., the size and shape of the injured portion on the body). It should be noted that more than one size and type of flexible wrap 80 may be included in the kit 70.

The operations discussed above with respect to the described methods may be performed in a different order from those described herein. It should be noted that attaching a patch to a body includes attaching the patch directly or indirectly to the body. In addition, FIGS. 1-6 are representational and are not necessarily drawn to scale. Certain proportions thereof may be exaggerated, while others may be minimized.

The systems and methods described herein provide a user or therapist with flexibility in determining the type of therapy to apply to an injured area of a body. The systems and methods also reduce the amount of therapy-related items that would otherwise need to be inventoried in order provide the ability to apply different kinds of therapy to an injured portion of a body.

While the invention has been described in detail with respect to the specific aspects thereof, it will be appreciated that those skilled in the art, upon attaining an understanding of the foregoing, may readily conceive of alterations to, variations of, and equivalents to these aspects which fall within the spirit and scope of the present invention, which should be assessed accordingly to that of the appended claims.

1. A system for providing therapy to a portion of a body, the system comprising:
a holder;
a first patch held by said holder, wherein said first patch applies a first therapy to the portion of the body when said first patch is placed near the portion of the body; and
a second patch held by said holder, wherein said second patch applies a second therapy to the portion of the body when said second patch is placed near the portion of the body.

2. The system of claim 1 further comprising a third patch held by said holder, wherein said third patch applies a third therapy to the portion of the body when said third patch is placed near the portion of the body.

3. The system of claim 2 wherein said first patch is an exothermic heating patch and said second patch is an endothermic cooling patch, and wherein said third patch is a topical analgesic patch.

4. The system of claim 1 wherein said first patch is an exothermic heating patch and said second patch is an endothermic cooling patch.

5. The system of claim 1 wherein said first patch is within a first enclosure and said second patch is within a second enclosure.

6. The system of claim 1 wherein said holder is a container.

7. The system of claim 1 wherein said holder is a board.

8. The system of claim 1 further comprising a plurality of first patches held by said holder.

9. The system of claim 1 wherein said first patch and said second patch are releasably attached to said holder.

10. The system of claim 1 further comprising a flexible wrap for securing at least one of said first patch and said second patch near the portion of the body.

11. A kit of parts for providing therapy to a portion of a body, the kit comprising:
a first patch adapted to apply a first therapy to the portion of the body when said first patch is placed near the portion of the body;
a second patch adapted to apply a second therapy to the portion of the body when said second patch is placed near the portion of the body; and
a holder adapted to hold said first patch and hold said second patch.

12. The kit of claim 11 wherein said holder is a container.

13. The kit of claim 11 wherein said first patch is an exothermic patch adapted to heat to the body and said second patch is an endothermic patch adapted to cool the body.

14. The kit of claim 11 wherein said first patch is within a first enclosure.

15. The kit of claim 14 wherein said first enclosure is a substantially air-tight enclosure.

16. The kit of claim 11 further comprising a flexible wrap adapted to secure at least one of said first patch and said second patch near the portion of the body.

17. The kit of claim 16 wherein said flexible wrap is adapted to secure both said first patch and said second patch near the portion of the body.

18. The kit of claim 11 wherein said first patch is adapted to be releasably secured to said holder and said second patch is adapted to be releasably secured to said holder.

19. The kit of claim 11 further comprising a third patch adapted to apply a third therapy to the portion of the body when said third patch is placed near the portion of the body.

20. The kit of claim 19 wherein said first patch is a heat patch and said second patch is a cooling patch, and wherein said third patch is an electrical stimulating device.

21. A method of providing therapy to a portion of a body, the method comprising:
selecting a patch from a holder that holds a plurality of patches, wherein at least one of the patches is adapted to apply a different type of therapy to the portion of the body than the other patches; and
applying the patch near the portion of the body.

22. The method of claim 21 further comprising determining an appropriate therapy to provide to the portion of the body.

23. The method of claim 21 wherein applying the patch near the portion of the body includes applying the patch to the portion of the body.

24. The method of claim 21 wherein selecting a patch from a holder that holds a plurality of patches includes selecting a heat patch from the plurality of patches.

25. The method of claim 21 wherein selecting a patch from a holder that holds a plurality of patches includes removing the patch from a container.

26. The method of claim 21 wherein selecting a patch from a holder that holds a plurality of patches includes releasing the patch from a board.

27. The method of claim 21 wherein applying the patch near the portion of the body includes wrapping the patch within a flexible wrap that is wrapped around the body.

28. The method of claim 21 wherein selecting a patch from a holder that holds a plurality of patches includes selecting at least two patches from the holder where one of the patches provides a different type of therapy than the other patches.

29. The method of claim 28 wherein applying the patch near the portion of the body includes applying the patches near the portion of the body to provide a combined therapy.

30. The method of claim 29 wherein applying the patches near the portion of the body to provide a combined therapy includes applying at least one topical analgesic patch and at least one transdermal analgesic patch.

31. The method of claim 29 wherein applying the patches near the portion of the body to provide a combined therapy includes applying at least one heat patch and at least one transdermal analgesic patch.
